(19) United States
(12) Patent Application Publication
(10) Pub. No.: US 2005/0031609 A1
(43) Pub. Date: Feb. 10, 2005

(54) Compositions for use in treating ige-associated disorders

(76) Inventors: Thomas Hultsch, Mountain Lakes, NJ (US); Jens Brauburger, Oberasbach (DE)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/476,263

(22) PCT Filed: May 11, 2001
(86) PCT No.: PCT/EP01/05412

(51) Int. Cl. A61K 39/395

(52) U.S. Cl. 424/131.1

(57) ABSTRACT

The present invention provides methods of treating IgE-associated disorders and products for use therein. The methods comprise administering to a subject an amount of a first composition comprising an immunogenie antigen and an amount of a second composition that inhibits the activity of IgE. The methods are particularly useful in treatment of allergies such as allergic rhinitis. These combination methods offer significant advantages, such as improving the efficacy of therapy while showing a good safety profile.

### TECHNICAL FIELD

The present invention provides methods of treating IgE-associated disorders and products for use therein. The methods are particularly useful in treatment of allergies such as allergic rhinitis.

### BACKGROUND OF THE INVENTION

Allergy is an altered state of immune reactivity, usually denoting hypersensitivity. Hypersensitivity reactions involve humoral mediators such as interleukins and interferons, complement proteins, and immunoglobulins. One of the most common pathologic features of allergic conditions is the presence of inflammation caused by activation of the immune system.

For an allergic reaction to occur, an individual must have had prior exposure to an allergen. Following the initial antigen exposure, the immune system produces IgE specific for the inciting antigen. The antigen-specific IgE then binds to mast cell membranes via IgE receptors. When re-exposed to the antigen, the antigen-specific IgE antibody binds to the antigen and activates the mast cells. Such mast cell activation causes a release of vasoactive and neuronal stimulatory mediators such as histamines, leukotrienes, prostaglandins, bradykinin, and platelet-activating factor and inflammatory mediators such as eosinophils, basophils, neutrophils, and CD4 T-lymphocytes.

Allergic rhinitis is a clinical disorder characterized by nasal congestion, rhinorrhea, sneezing, and itching. Severity of these symptoms can vary from year to year, with occasional spontaneous remissions. Therefore, allergic rhinitis is classified by whether symptoms occur during certain seasons (SAR or seasonal allergic rhinitis) or year-round (PAR or perennial allergic rhinitis). The seasonal variety is usually caused by pollens from plants that depend on the wind for cross-pollination, such as grasses, trees, weeds, and mold spores.

Serious complications, such as nasal polyps, recurrent sinusitis, recurrent ear infections, and hearing loss, can occur if allergic rhinitis is not treated or is undertreated. Psychosocial effects can include frequent absences from work or school, poor performance, poor appetite, malaise, and chronic fatigue.

Allergic asthma as a clinical disorder that is characterized by three components: airway inflammation; airway obstruction, which is reversible; and increased sensitivity, referred to as hyperreactivity. Obstruction to airflow is measured by a decrement in forced expired volume in one second (FEV I) which is obtained by comparison to baseline spirometry. Hyperreactivity of the airways is recognized by decreases in FEVI in response to very low levels of histamine or methacholine. Hyperreactivity may be exacerbated by exposure of the airways to allergen.

Generally, an optimal treatment for allergy would reduce or remove the symptoms and also correct the immune system's abnormal reactions. Use of symptomatic drugs such as antihistamines or steroids can reduce symptoms, but they do not deal with the underlying disease.

Specific immunotherapy, which is also known as specific allergy vaccination, desensitization or hyposensibilisation, is a treatment option that interferes with the basic mechanisms of the allergic disease. Specific immunotherapy is used for respiratory allergies—e.g. tree pollens, grass pollens, animal dander, moulds and house dust mites. It is also effective as protection against severe allergic reactions to bee and wasp stings. Regular vaccination with minute quantities of the offending allergen in gradually increasing doses stimulates the immune system to develop an increased tolerance.

In view of the above-described advantages of specific immunotherapy, it is highly desirable to further increase the efficacy of this therapeutical option in allergic disorders, while maintaining or even improving the safety profile of specific immunotherapy.

### SUMMARY OF THE INVENTION

The present invention now provides a method of treating a subject having an IgE associated disorder comprising administering to the subject an amount of a first composition comprising an immunogenic antigen and administering to the subject an amount of a second composition that inhibits the activity of IgE.

In another aspect of the invention there is provided the use of a composition that inhibits the activity of IgE for the manufacture of a medicament for the treatment of a subject having an IgE associated disorder, wherein the subject is treated simultaneously or sequentially with a composition comprising an immunogenic antigen.

In yet another aspect of the invention products are provided which contain a composition comprising an immunogenic antigen and a composition that inhibits the activity of IgE as a combined preparation for simultaneous, separate or sequential us in the therapy of an IgE associated disorder.

Also within the scope of this invention is a pharmaceutical formulation comprising a composition that inhibits the activity of IgE and a composition comprising an immunogenic antigen.

Furthermore, there is provided a method of treating an allergic response to an antigen or allergy-related disorder during antigen-specific immunotherapy of a subject comprising administering to the subject an amount of a first composition that inhibits the activity of IgE sufficient to decrease the activity of IgE in the subject and administering to the subject a second composition comprising an amount of the antigen sufficient to modulate the immune response to the antigen.

### DETAILED DESCRIPTION THE INVENTION

All of the cited literature included in the preceding section, as well as the cited literature included in the following disclosure, are incorporated herein by reference.

The present invention provides novel methods of treating a subject having an IgE associated disorder. This combination method comprises administering to the subject an amount of a first composition comprising an immunogenic antigen and administering to the subject an amount of a second composition that inhibits the activity of IgE.

The term “treatment” as used herein includes alleviation of one or more symptoms of the disorder, diminishment of the extent of the disorder, stabilization of the disorder, delay or slowing of disorder progression, amelioration or palliation of the disorder, and partial or total remission. Treatment also includes prolonging survival as compared to expected survival if not receiving treatment. The methods of the invention are appropriate for prevention of an allergic response as well as treating a pre-existing allergic condition.

The method of treatment of the invention particularly relates to clinical methods known as specific immunotherapy or desensitization. Specific immunotherapy refers to the process of administering increasing doses of an antigen, such as, in particular, an allergen to which the subject has demonstrated sensitivity. Examples of allergen doses used for desensitization are known in the art and are further described in the Examples hereinbelow.

Generally, the treatment provided by the present invention may be short-term pre-seasonal treatment or may last for several years, as, for example, with vaccinations in alternate months. The first and second compositions of the invention can, for example, be given as injections. It is also possible to place the allergen extract as small drops under the tongue, for example, two to three times a week.

Until the immune system responds there may still be need to continue with the medication. Usually, after treatment of about two to about six months, the need for drugs will decrease as the symptoms will become less severe. The effect may be maintained for several years, in particular up to 5-10 years or more, after the treatment has been completed. The natural aggravation of the allergic disease may be inhibited and the development of asthma and/or new allergies may be prevented by the method of treatment according to the invention.

An “IgE associated disorder” within the meaning of the invention is a condition which is characterized by elevated IgE levels. The elevated IgE levels may or may not be persistent. IgE associated disorders include, but are not limited to, allergy and allergic reactions, asthma, rhinitis, conjunctivitis, urticaria, shock, hymenoptera sting allergies, drug allergies, and parasite infections. The term also includes related manifestations of these disorders.

In a preferred embodiment the IgE associated disorder is an allergy,

An allergy is a disorder characterized by an allergic response to antigen, in particular it is characterized by the generation of antigen-specific IgE and the resultant effects of the IgE antibodies. As is well-known in the art, IgE binds to IgE receptors on mast cells and basophils. Upon later exposure to the antigen recognized by the IgE, the antigen cross-links the IgE on the mast cells and basophils causing degranulation of these cells.

In a preferred embodiment allergy is allergic asthma, allergic rhinitis, and, in particular, perennial allergic rhinitis (PAR) and seasonal allergic rhinitis (SAR). SAR is a particularly preferred indication for treatment by the methods of the invention. For example, in one particularly preferred embodiment the IgE associated disorder is SAR in patients having an age of 6-17 years. Also preferred are young patients having an age of 6-12 years, 6-10 and 6-8 years. Also preferred are patients having a clinical history below 2 years of moderate to severe SAR. Furthermore, preferred are patients having a serum IgE level between 30 and 1300 IU/ml.

Seasonal allergic rhinitis is a form of allergic rhinitis that shows seasonal variety. In contrast, in perennial allergic rhinitis, symptoms occur throughout the year. However, a pollen allergy can contribute to seasonal exacerbations of rhinitis in patients with perennial symptoms.

The term “immunogenic antigen” according to the invention means a substance that is recognized and bound specifically by an antibody or by a T cell antigen receptor. Such an antigen may preferredly be an allergen as defined hereinbelow. Haptens are immunogenic antigens within the meaning of the invention. A hapten is a low molecular weight compound that is not immunogenic by itself but is rendered immunogenic when conjugated with an immunogenic molecule containing antigenic determinants.

In a preferred embodiment of this invention the antigen is capable of eliciting or modulating an immune response in a human being as measured by techniques know in the art. Such tests of immune responses are known to the person skilled in the art, in particular skin tests and tests specifically assaying the IgE levels are useful to quantify an immune response. An immune response is elicited if there was no prior immune response to said antigen, it is modulated if it significantly changes as measured by the respective test. A change may be significant for example if increased or decreased by at least 10%, 20%, 50% or even 2 fold. Immunogenic antigens capable of eliciting or modulating an immune response in a human being generally can include peptides, proteins, glycoproteins, polysaccharides, gangliosides and lipids; portions thereof and combinations thereof. The antigens can be those found in nature or can be synthetic.

In a preferred embodiment of the invention the antigen is an allergen. The term “allergen” means an antigen or antigenic portion of a molecule which elicits an allergic response upon exposure to a subject. Typically the subject is allergic to the allergen as can be measured by clinical tests, assessed by taking the clinical history of the subject or any other suitable method known in the art and as further described in the Examples hereinbelow. An antigen is said to be an allergen if only a small subset of subjects exhibit an immune response upon exposure to the molecule. Numerous isolated allergens are known in the art. For example, common allergens in patients with seasonal allergic rhinitis include pollen from grasses, trees, weeds and mold spores. Common allergens in patients with perennial allergic rhinitis are household dust mites, wood dust, molds, fungus spores, feather pillows, animal dander, animal hair, and cigarette smoke. the most

In a preferred embodiment of the invention the allergen is an aeroallergen. In a particularly preferred embodiment of the invention the aeorallergen is a grass pollen allergen, such as for example ALK SQ as further described in the Examples hereinbelow.

Further useful allergens are, for example, bee-venom extracts, dust mite extracts and rhagweed extracts.

A composition that inhibits the activity of IgE Is a composition that contains at least one agent that reduces IgE activity when compared to otherwise same conditions, except for the absence of the composition. IgE activity may be measured by the circulating levels of IgE, but can also be measured by activities associated with IgE function, such as binding to basophils, anaphylaxis, and binding to receptors such as Fc receptors.

Generally, compositions that inhibit the activity of IgE may include, for example, anti-IgE antibodies, IgE receptors, anti-IgE receptor antibodies, variants of IgE antibodies, ligands for the IgE receptors, and fragments thereof. Variant IgE antibodies may have amino acid substitutions or deletions at one or more amino acid residues.

In a preferred embodiment the composition that inhibits the activity of IgE comprises an anti-IgE antibody. Preferredly the anti-IgE antibody is a humanized murine antibody or a fully human antibody. Most preferredly the anti-IgE antibody is Omalizumab, which is also named “E25”. Another preferred anti-IgE antibody is named “E26” as further defined hereinbelow.

Anti-IgE antibodies are described in the prior art, and in greater detail in the International applications WO 93/04173 and WO 99/01556. WO 99/01556 specifically describes Omalizumab, also named E25, in FIG. 12, and in the sequences ID-No. 13-14. Antibody molecules comprising a E26 sequence are described in WO 99/01556 and are selected from the group of F(ab) fragment (Sequence ID Nos. 19-20), sFv fragment (Sequence ID No. 22) and F(ab)′₂ fragment (Sequence Nos. 24-25), in accordance to FIGS. 12-15. Within this invention, the terms E25 and E26 shall be construed accordingly. Preferably, the IgE antibodies of the instant invention do not result in histamine release from mast cells or basophils.

Furthermore, U.S. Pat. No. 5,449,760 generally describes anti-IgE antibodies that bind soluble IgE but not IgE on the surface of B cells or basophils. Antibodies such as these bind to soluble IgE and inhibit IgE activity by, for example, blocking the IgE receptor binding site, by blocking the antigen binding site and/or by simply removing the IgE from circulation. Additional anti-IgE antibodies and IgE-binding fragments derived from the anti-IgE antibodies are described in U.S. Pat. No. 5,656,273. U.S. Pat. No. 5,543,144 describes anti-IgE antibodies that bind soluble IgE and membrane-bound IgE on IgE-expressing B cells but not to IgE bound to basophils.

Generally, the compositions of the invention are administered in therapeutic amounts. The term “therapeutic amount” as used herein generally denotes an amount that prevents or ameliorates symptoms of a disorder or responsive pathologic physiological condition. For example, in a preferred embodiment of the invention the allergen is administered in an amout sufficient to induce desensitization to the allergen in combination with the composition that inhibits the activity of IgE. This amount may or may not be an amount that is therapeutic in the absence of the composition that inhibits the activity of IgE.

Generally, the “therapeutic amount” of a substance or composition depends upon the context in which it is being applied. In the context of administering a composition that inhibits IgE activity, a therapeutic amount is an amount sufficient to achieve any such inhibition, which need not be total. A therapeutic amount can be administered in one or more administrations, and it is understood that, especially in the context of allergy desensitization therapy, a therapeutic amount is achieved over a series of administrations, typically in increasing dosages.

In a preferred embodiment of the invention the median symptom load is reduced by at least 10%, preferredly by at least 20% or even by at least 40%. The symptom load is the mean daily symptom score plus mean daily rescue medication score as defined in the Examples below.

In another preferred embodiment of the invention the days with intake of any allergy medication are reduced by at least 10%, preferredly by at least 20% or even by at least 60%. For example, such reduction can be achieved in the birch and/or in the grass pollen season.

In another preferred embodiment of the invention the median use of rescue medication is reduced by at least 10%, preferredly by at least 20% or even by at least 60%. Most preferred is a reduction above 70%. For example, such reduction can be achieved in the birch and/or in the grass pollen season.

In the practice of the invention, the first and second compositions can be administered to the subject in a pre-determined order or/and simultaneously. In particular, the first composition including the antigen may be administered before the second composition. In a preferred embodiment the first composition is administered with the second composition. Preferredly, before first composition is administered with the second composition, there has been a pre-treatment with the first composition.

The present invention also provides for a method wherein in a first treatment period the first composition is titrated up to a maintenance dose, and in a second treatment period the second composition is administered in addition to the maintenance dose of the first composition. For example, in one preferred embodiment the first treatment period may be about 12 weeks and the second treatment period may be about 24 weeks. In one preferred embodiment the first treatment period is started at least 14 weeks prior to the relevant allergen season, such as for example the relevant pollen season. Preferredly, there is no time interval between the two treatment periods.

Also provided by this invention is a method wherein the efficacy of treatment is monitored by the measurement of one or more surrogate markers during the treatment period. Suitable surrogate markers are, for example, leukotriens, markers for the activation of mast cells, such as, for example, tryptase, and eosinophil counts.

The present invention also provides products containing a composition comprising an immunogenic antigen and a composition that inhibits the activity of IgE as a combined preparation for simultaneous, separate or sequential use in the therapy of an IgE associated disorder.

Further, as would be readily understood by one skilled in the art, the active ingredients described in any of the embodiments herein may be combined into a single composition for simultaneous administration of one or more of the active ingredients.

Accordingly, the present invention also provides a pharmaceutical formulation comprising a composition that inhibits the activity of IgE and a composition comprising an immunogenic antigen. Such a formulation will be prepared according to methods know in the art and will dependent on the nature of the active agents in the first and second composition. In particular, such formulations may advantageously include buffering agents, preservatives, stabilizers, and non-ionic surfactants or detergents.

Buffering agents help to maintain the pH in the range which approximates physiological conditions. They are preferably present at concentration ranging from about 2 mM to about 50 mM. Suitable buffering agents for use with the present invention include both organic and inorganic acids and salts thereof such as citrate buffers (e.g., monosodium-citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.). tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium famarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be mentioned phosphate buffers, histidine buffers and trimethylamine salts such as Tris.

Preservatives are added to retard microbial growth, and are added in amounts ranging from 0.2%-1% (w/v). Suitable preservatives for use with the present invention include phenol, benzyl alcohol, meta-cresol, methyl paraben, propyl paraben, octadecyl dimethylbenzyl ammonium chloride, benzalconium halides (e.g., chloride, bromide, iodide), hexamethonium chloride. alkyl parabens such as methyl or propyl paraben, catechol, resorcinol, cyclohexanol, and 3-pentanol.

Isotonicifiers sometimes known as “stabilizers” are present to ensure isotonicity of liquid compositions of the present invention and include polhydric sugar alcohols, preferably trihydric or higher sugar alcohols, such as glycerin, erythritol, arabitol, xylitol, sorbitol and mannitol. Polyhydric alcohols can be present in an amount between 0.1% to 25% by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhyric sugar alcohols (enumerated above); amino acids such as arginine, lysine, glycine, glutamine, asparagine, histidine, alanine, omithine, L-leucine, 2-phenylaianine, glutamic acid, threonine, etc., organic sugars or sugar alcohols, such as lactose, trehalose. stachyose, mannitol, sorbitol, xylitol, ribitol, myolnisitol, galaakol glycerol and the likq including cyditols such as inositol; polyethylene glycol; amino acid polymers; sulfur containing reducing agents, such as urea, glutathione, thioctic acid, sodium thloglycolate, thioglycerol, α-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffmose; polysaccharides such as dextran. Stabilizers are present in the range from 0. 1 to 10,000 weights per part of weight active protein.

Non-ionic surfactants or detergents (also known as “wetting agents”) are present to help solubilize the therapeutic agent as well as to protect the therapeutic protein against agitation-induced aggregation, which also permits the formulation to be exposed to shear surface stressed without causing denaturatlon of the protein. Suitable non-ionic surfactants include polysorbates (20, 80, etc.), polyoxamers (184, 188 etc.), Pluronice polyols, polyoxyethylene sorbitan monoethers (TweenO-20, TweenO-80, etc.). Non-ionic surfactants are present in a range of about 0.05 mg/ml to about I I mg/mL preferably about 0.07 mg/ml to about 0.2 mg/ml. Additional miscellaneous excipients include bulking agents, (e.g. starch), chelating agents (e.g. EDTA), antioxidants (e.g., ascorbic acid, methionine, vitamin E), and cosolvents. The formulation herein may also contain more than one active compound as necessary for the particular indication being treated, preferably those with complementary activities that do not adversely affect each other. For example, it may be desireable to further provide an immunosuppressive agent. Such molecules are suitably present in combination in amounts that are effective for the purpose intended.

The active ingredients may also be entrapped in microcapsule prepared, for example, by coascervation techniques or by interfacial polymerization, for example, hydroxymethyl cellulose or gelatin-microcapsule and poly-(methylmethacylate) microcapsule, respectively, in colloidal drug delivery systems (for example, liposomes, albumin micropheres, microemulsions. nano-particles and nanocapsules) or in macroemulsions. Such techniques are disclosed in Remington˜Pharmaceutical Sciences, 16th edition, A. Osal, Ed. (1980). The formulations to be used for in vivo administration must be sterile. This is readily accomplished, for example, by filtration through sterile filtration membranes.

Sustained-release preparations may be prepared. Suitable examples of sustained-re lease preparations include semi-permeable matrices of solid hydrophobic polymers containing the antibody mutant, which matrices are in the Am of shaped articks, e.&, fikv or microcapsules. Examples of sustained-release matrices include polyesters, hydmgels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(+3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37'C, resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, iyophilizing from acidic solutions, controlling moisture content, using appropriate additives, and developing specific polymer matrix compositions.

Also within the scope of this invention is the use of a composition that inhibits the activity of IgE for the manufacture of a medicament for the treatment of a subject having an IgE associated disorder, wherein the subject is treated simultaneously or sequentially with a composition comprising an immunogenic antigen.

Also within the scope of this invention are methods and composition as described in patent application WO00/16804 (Dynavax). WO00/16804 is explicitly incorporated for its relevant disclosure regarding the methods and compositions described in this paragraph. Accordingly this invention also provides a method of treating an allergic response to an antigen or allergy-related disorder during antigen-specific immunotherapy of a subject comprising administering to the subject an amount of a first composition that inhibits the activity of IgE sufficient to decrease the activity of IgE in the subject and administering to the subject a second composition comprising an amount of the antigen sufficient to modulate the immune response to the antigen. In one embodiment of this method the composition that inhibits the activity of IgE comprises an anti-IgE antibody. Also provided is a composition comprising an antigen for use in immunotherapy according to this method, wherein the antigen is at a concentration higher than acceptable for use in allergy desensitization therapy. Also provided is a a kit comprising this composition in suitable packaging.

### EXAMPLES

### Example 1

### Omalizumab Combined with Specific Immune Therapy (SIT) in Seasonal Allergic Rhinitis

This study (“D01”) was designed to show safety and efficacy of omalizumab in combination with specific immunotherapy in children and adolescents 6-17 years old with SAR. The study rational postulated that the combination of an active vaccination (SIT) plus a passive vaccination (anti-IgE) should have an additive effect.

Study D01 was a phase III, placebo-controlled, multicenter, clinical study. Children and adolescents with sensitization to birch and grass pollens suffering from seasonal allergic rhinitis were randomized into four groups: either birch or grass pollen SIT (SIT-birch; SIT-grass) in combination with either omalizumab or placebo. Treatment was started in winter 1999 and was continued during the 2000 pollen season by subcutaneous injections. Dosage of omalizumab was adjusted depending on baseline IgE level and body weight.

The results demonstrate that omalizumab, administered using the same dosing scheme as for allergic asthma (based on patients baseline total IgE level and body weight) was safe and effective for the treatment of SAR and for the combination with SIT. Omalizumab reduced the symptoms of SAR (nose and eyes), the use of rescue medication (topical and systemic) significantly over SIT alone, which is currently best medical practice. Consequently, the symptom load (prim. efficacy endpoint: mean daily symptom score plus mean daily rescue medication score) was reduced significantly in the SIT plus omalizumab group versus SIT alone group.

Omalizumab was well tolerated and showed an excellent safety profile over the 24 weeks treatment period. No case of anaphylaxis or an anaphylactoid reaction was observed. There was no significant incidence of urticaria in any treatment group. In vitro assays provide additional evidence for suppression of allergic reaction in vivo (tryptase, ECP).

Patient Population and Study Design (Study D01)

Study D01 was a 36-week double blind, placebo-controlled, randomized, multi-center, parallel group study. The study enrolled a total of 225 patients age 6-17 years. Before start of pollen season 3 patients discontinued the study prematurely due to protocol violations and have never received omalizumab/placebo. Therefore the safety sample consists of 222 patients, of which 132 belonged to the age group 6-12 years. Because one patient received study medication only once and discontinued the study thereafter before start of birch pollen season and before any measurement of efficacy parameter this patient was excluded from the intent-to-treat (ITT) sample. Efficacy was analyzed for all those patients of the ITT sample (N=221), of which 131 belonged to the age group 6-12 years. All patients suffered from SAR due to birch and grass pollen. SIT (current standard therapy) was administered to all patients, for either birch or grass according to the instructions of the manufacturer. During the first 12 weeks (pre-seasonal) SIT therapy was titrated up to maintenance dose. Thereafter, but at least 2 weeks prior to start of birch season, omalizumab or placebo was added for 24 weeks at the dose resulting from the asthma-dosing table as described hereinbelow. Safety was assessed for the 24-week omalizumab treatment period; efficacy was assessed for the pollen seasons as defined by pollen counts locally.

In case of an overlap of both pure pollen seasons the entire pollen season was defined as the first day of the birch pollen season until the last day of the grass pollen season. If there was an intermediate interval between both pollen seasons this interval was excluded from the entire pollen season, i.e. the entire pollen season was the interval from the first day of the birch pollen season until the last day of this season and the interval of the first day of the grass pollen season until the last day in this season.

Patients were randomized to receive SIT for either birch or grass pollen beginning treatment at least 14 weeks prior to the pollen season. Additionally, patients received either subcutaneous omalizumab or placebo for 24 weeks during the entire birch and grass pollen season. Daily symptom scores (nose and eyes) and rescue medication usage (antihistamines, corticosteroids) were assessed.

The patient population included children and adolescents aged 6-17 years who suffered from moderate to severe symptoms of SAR. Patients had to meet the following inclusion criteria: (a) serum IgE levels between 30-1300 IU/ml, (b) Positive IgE reactivity (CAP≧2) for birch and grass pollen, (c) clinical history of 2 or more years of moderate to severe SAR (birch and grass).

The inclusion criteria were:
1. Male and female patients aged ≧6 and <18 years
2. Patients must have a clinical history of two or more years of moderate to severe seasonal birch and grass allergic rhinitis
3. Patients must have positive IgE reactivity (CAP≧2) for birch and grass pollen at randomization or in the three months prior to randomization visit.
4. Patients must be asymptomatic or minimally symptomatic during the month before the start of the birch pollen season. Patients could be minimally symptomatic during hazel or alder pollen seasons.
5. Baseline FEV-1≧70% of the predicted normal value for the patient within 3 month prior to or at randomization. This criterion for FEV-1 must be demonstrated 6 or more hours after short-acting beta-2-agonist use or 72 hours or more after long-acting beta-2-agonist use
6. Patients must have a baseline serum IgE level ≧30 IU/ml and ≦1300 IU/ml and a corresponding body weight
7. Patients must meet pretrial eligibility requirements for trial enrollment (acceptable medical history, physical examination results, and acceptable laboratory test results)
8. Patients must weigh ≦100 kg at the time of enrollment
9. A signed Informed Consent prior to initiation of trial procedures

The exclusion criteria were:
1. Patients with clinical relevant allergy for perennial allergens with clinical relevance (e.g. stuffy nose due to house dust mite). Note: Patients with sensitization to environmental allergens could be included if this is not a clinical relevant allergy.
2. Patients with a history of severe anaphylactoid or anaphylactic reaction(s)
3. Patients with a history of perennial asthma with corresponding durable treatment with inhaled and/or systemic steroids
4. Patients with a history of immunotherapy to treat (birch/hazel/alder) or (grass/rye) SAR during the previous five years
5. Patients with known hypersensitivity to any ingredients, including excipients (sucrose, histidine, and polysorbate 20), of rhuMAb-E25 or related drugs (i.e. monoclonal antibody, polyclonal gammaglobulin)
6. Patients with known hypersensitivity to the trial rescue medication or related drugs
7. Patients using Montelukast (Singulair®) Zafirlukast (Accolate®) or other leukotriens antagonists and Zileuton (Zyflo®) or other 5-lipoxygenase enzyme inhibitors within 7 days prior to randomization visit and during this trial
8. Patients taking cromolyn sodium (DNCG) or nedocromil sodium (inhaled, nasal or eye drops) within 7 days of randomization and during this trial
9. Patients previously exposed to rhuMAb-E25
10. Patients with active or a recent history (<1 months) of any of the following types of rhinitis: Perennial non-allergic rhinitis, topical or systemic rhinitis medicamentosa, vasomotor rhinitis, structurally related disease (for example, severe deviated nasal septum)
11. Patients with a history of acute infectious sinusitis in the previous month
12. Patients with chronic heart or lung disease (emphysema, cor pulmonale, irreversible damages due to long standing bronchitic symptoms, chronicle airway disease with corresponding inflammatory changes in mucosa and irreversible hyperreactivity, bronchiectasis). Patients with another significant systemic disease or a history of such disease. Patients suffering from a primary or secondary immune disease (e.g. AIDS). Patients with known parasitic infections
13. Patients taking beta-adrenergic antagonist medications regularly (e.g., propranolol)
14. Patients taking tricyclic anti-depressants or monoamine-oxidase inhibitors regularly
15. Patients using antihistamines (e.g. chlorpheniramine, acrivastine, promethazine, tripelennamine, diphenhydramine, terfenadine, fexofenadine or other “short-acting” antihistamines, hydroxyzine, loratadine, clemastine or long-acting antihistamines, i.e. astemizole), within 1 month of randomization visit and during this trial. Note: Zyrtec® (Cetirizine) and Livocab® (levocabastine hydrochloride) are rescue medication for this trial and therefore not excluded during double blind treatment period.
16. Patients taking oral, intramuscular, and intravenous steroids within 1 month of randomization visit, or inhaled nasal steroids within 15 days of randomization visit, and at any time during the trial. Note: Prednisolon (Decortin® 50) is rescue medication for this trial and therefore not excluded during double blind treatment period.
17. Patients taking systemic immune suppressive medication (e.g. ciclosporine) within 1 month of randomization visit and during this trial
18. Patients taking ACE inhibitors within 1 month of randomization visit
19. Treatment with an experimental, non-approved drug, or investigational drug within 1 month of randomization visit and during this trial
20. Patients previously randomized into the trial
21. Patients with travel plans for more than 14 connected days outside of Germany during the pollen seasons.
22. Pregnant women, nursing mothers or women of child bearing potential, who do not use a reliable contraceptive method. Any patient becoming pregnant during the course of the trial must be discontinued and followed up until resolution of pregnancy.
23. Patients with a history of noncompliance to medical regimens and patients who are considered potentially unreliable
24. Other reasons by assessment of the investigator, which make patients participation into the trial not appropriate

The recruited population showed the demography and baseline characteristics of table 1 (ITT sample) and table 2 (Safety sample):

TABLE 1
Demography and baseline characteristics/ITT sample

| | omalizumab | Placebo |
|---|---|---|
| | (n = 114) | (n = 107) |
| Age [ys] | | |
| mean ± SD | 12.0 ± 3.1 | 11.5 ± 3.0 |
| median (min-max) | 12 (6-17) | 12 (6-17) |
| Sex [%] male | 51.8 | 64.5 |
| Duration of SAR [ys] | | |
| mean ± SD | 6.4 ± 2.9 | 6.0 ± 3.0 |
| median (min-max) | 6.0 (3-2) | 5.0 (2-2) |
| Serum IgE [IU/ml] | | |
| mean ± SD | 423.3 ± 257.4 | 382.7 ± 235.5 |
| median (min-max) | 345.5 (45.0-1030.0) | 337.0 (31.6-998.0) |
| Serum spec. IgE birch [IU/ml] | | |
| mean ± SD | 23.3 ± 33.5 | 25.64 ± 37.9 |
| median (min-max) | 7.5 (0-125.0) | 6.4 (0-125.0) |
| Serum spec. IgE grass [IU/ml] | | |
| mean ± SD | 71.1 ± 50.0 | 65.0 ± 49.9 |
| median (min-max) | 74.6 (0.9-125.0) | 54.8 (0-125.0) |
| Asthma history [%] yes | 15 | 17 |

TABLE 2
Demography and baseline characteristics/Safety sample

| | Omalizumab | Placebo |
|---|---|---|
| | (n = 114) | (n = 108) |
| Age [ys] | | |
| mean ± SD | 11.95 ± 3.14 | 11.51 ± 3.00 |
| median (min-max) | 12 (6-17) | 12 (6-17) |
| Sex [%] male | 51.8 | 63.9 |
| Duration of SAR [ys] | | |
| mean ± SD | 6.4 ± 2.9 | 6.0 ± 3.0 |
| median (min-max) | 6.0 (3-2) | 5.0 (2-2) |
| Serum IgE [IU/ml] | | |
| mean ± SD | 423.3 ± 257.4 | 381.9 ± 234.5 |
| median (min-max) | 345.5 (45.0-1030.0) | 333.0 (31.6-998.0) |
| Serum spec. IgE birch [IU/ml] | | |
| mean ± SD | 23.3 ± 33.5 | 25.4 ± 37.8 |
| median (min-max) | 7.5 (0-125.0) | 6.1 (0-125.0) |
| Serum spec. IgE grass [IU/ml] | | |
| mean ± SD | 71.1 ± 50.0 | 65.6 ± 50.0 |
| median (min-max) | 74.6 (0.9-125.0) | 55.6 (0-125.0) |
| Asthma history [%] yes | 15 | 17 |

(For analysis of serum spec. IgE birch and grass: >100 were replaced by 125 and <0.35 was replaced by 0)

Efficacy parameter scores: Mean and median daily symptom scores were calculated based on the patients diary assessment of clinical symptoms. Symptoms were categorized into 7 domains (stuffy nose, runny nose, itchy nose, sneezing and itchy eyes, watery eyes, red eyes). Each category could score 0-3 (none-mild-moderate-severe). Daily rescue medication scores given were: 0 for no medication; 1 for topical antihistamines; 2 for systemic antihistamines, 3 for oral or topical corticosteroids. Only maximal score per day was assessed.

Efficacy parameter endpoints: The primary outcome variable was the symptom load (mean daily symptom score plus mean daily rescue medication score).

The secondary clinical efficacy variables measured were: symptom score (mean of the daily symptom score), rescue medication score (mean of the daily rescue medication score during entire pollen season), proportion of days with rescue and/or concomitant medication use, investigators global evaluation of treatment tolerability.

Safety assessments included monitoring and recording of all adverse events and serious adverse events, hematological, serum chemistry and urinary laboratory evaluations.

The study was conducted in Germany during the whole birch and grass pollen season 2000 in accordance with the protocol at all participating centers (17 German centers). Confirmatory efficacy analysis was performed for the ITT sample, safety analysis was done for the safety sample. Primary efficacy was analyzed for the per protocol sample (PP sample: 109 omalizumab, 98 placebo) additionally. In case of one of the following protocol deviations patients were excluded form the PP sample:

TABLE 3
Violations

| | | Number of violations | |
|---|---|---|---|
| Violation | | omalizumab | placebo |
| Compliance to SIT therapy | exception: | 1 | 1 |
| during monotherapy | premature discont. | | |
| phase <80% | of treatment or | | |
| Compliance to SIT therapy | study due to medical | 1 | 0 |
| during treatment | reason | | |
| comparison phase <80% | | | |
| Compliance to | | 0 | 0 |
| omalizumab/placebo <80% | | | |

| | | |
|---|---|---|
| PK/PD data shows that patient received at least | 4 | 8 |
| once omalizumab instead of placebo or placebo | | |
| instead of omalizumab | | |

A total of 225 patients were randomized (116 to omalizumab and 109 to placebo), 221 patients were analyzed with respect to efficacy (114 omalizumab, 107 placebo) of whom 219 (99%) completed the study.

Drug Treatment

rhuMAb-E25 is supplied as a sterile, freeze dried preparation that can be reconstituted to a final rhuMAb-E25 concentration of 125 mg/ml. Each 10 ml vial contains 208 mg rhuMAb-E25. rhuMAb-E25 must be stored refrigerated at (2°-8° C.) until time of administration to the subject, do not freeze. Each vial is reconstituted with 1.3 ml of Sterile Water for Injection (SWI), and the contents are gently swirled for 30 seconds, then left for up to 5 minutes to solubilize. 1.2 ml is then drawn up to deliver 150 mg of rhuMAb-E25. The formulation does not contain a preservative and is to be used for single-dose administration only.

After reconstitution, patients randomized to rhuMAb-E25 receive blinded test drug administered on a two or four weekly basis, dependent on baseline IgE levels. The corresponding placebo group receive placebo on a two or four weekly basis, dependent on IgE levels.

rhuMAb-E25 is administered using a disposable 25 gauge needle and a disposable plastic tuberculin-type syringe. The injections are administered in the deltoid region on the right arm. Alternately, the injections can be administered in the right thigh if medically significant reasons preclude administration in the deltoid region. The injections are administered subcutaneously.

The SIT hazel/alder/birch or grass/rye is titrated with ALK SQ up to the maintenance dose within 12 weeks followed by 4-weekly maintenance dose until the end of grass season. The dose may be adjusted as judged by the investigator according to the guidelines from ALK. After loading SIT into a tuberculin-type syringe SIT is matching to each other.

Dose interval and number of doses: 12 weeks of SIT titration with allergens from ALK is adequate to increase allergen doses to maintenance dose according to current guidelines of ALK for SIT.

The dose of rhuMAb-E25 which is based on baseline free serum IgE levels, is designed to suppresses free serum IgE to levels below 25 ng/ml. The data from previous trials have shown a significant reduction of symptoms in allergic patients when baseline serum free IgE levels were at or below 25 IU/ml. No modification in the drug concentration to suppression relationship was shown to occur after repeated dosing but baseline IgE concentration was identified as an important factor influencing dose.

The use of rescue medication, levocabastine hydrochloride for symptoms of nose, eye (Livocab® Kombi) and salbutamol (Sultanol® N) for symptoms of the lower airways, and if still uncontrolled, cetirizine (Zyrtec®), and if symptoms are still uncontrolled oral prednisolone (Decortin®) is permitted, as necessary, to control symptoms of severe allergic rhinitis.

TABLE 4
rhuMab-E25 Dosing Schedule
Number of injections per dose (mg)

| Dose | Number | Injection volume |
|---|---|---|
| (mg) | of injections | (mL) |
| 150 | 1 | 1.2 |
| 225 | 2 | 1.8 (1.2 + 0.6) |
| 300 | 2 | 2.4 (1.2 + 1.2) |
| 375 | 3 | 3.0 (1.2 + 1.2 + 0.6) |

TABLE 5
rhuMAb-E25 doses, SQ Administration

| Baseline | Milligrams (mg) Per Dose | | | | | | |
|---|---|---|---|---|---|---|---|
| IgE | Body weight (kg) | | | | | | Frequency of |
| (IU/mL) | 20-30 | >30-40 | >40-50 | >50-60 | >60-70 | >70-90 | Dosing |
| >30-100 | 150 | 150 | 150 | 150 | 150 | 150 | Q4wk |
| >100-200 | 150 | 150 | 300 | 300 | 300 | 300 | |
| >200-300 | 150 | 300 | 300 | 300 | 225 | 225 | Q2wk |
| >300-400 | 300 | 300 | 225 | 225 | 225 | 300 | |
| >400-500 | 300 | 225 | 225 | 300 | 300 | 375 | |
| >500-600 | 300 | 225 | 300 | 300 | 375 | | Not Dosed |
| >600-700 | 225 | 225 | 300 | 375 | | | |
| >700-800 | 225 | 300 | 375 | | | | |
| >800-900 | 225 | 300 | 375 | | | | |
| >900-1000 | 300 | 375 | | | | | |
| >1000-1100 | 300 | 375 | | | | | |
| >1100-1200 | 300 | | | | | | |
| >1200-1300 | 375 | | | | | | |

Efficacy Results

Efficacy of omalizumab treatment in this study population translates clinically in reduction of rescue medication intake (antihistamines and corticosteroids) and/or reduction of clinical symptoms.

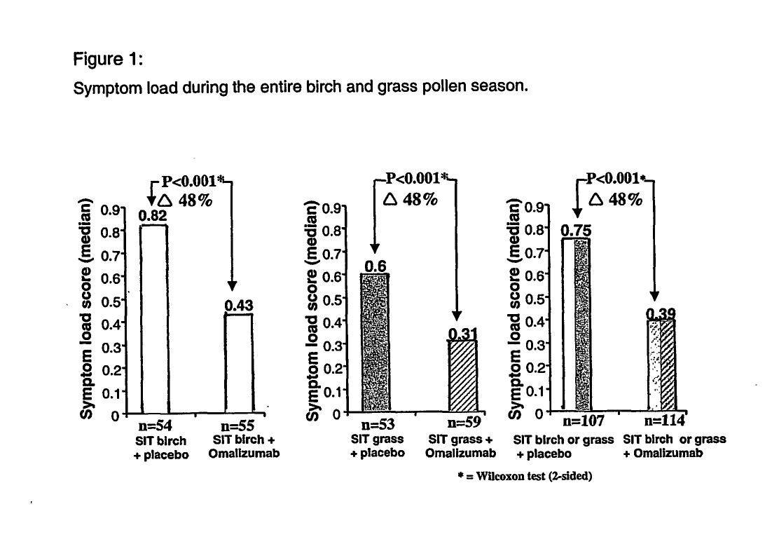

The median symptom load for patients treated with omalizumab was 48% lower than for patients treated with placebo (median 0.39 vs. 0.75, p<0.001; FIG. 1). The same pattern appeared for symptom score and rescue medication score. The response in the sub-group aged 6-12 ys was comparable to that in the analysis of all patients.

The results demonstrate that Xolair is effective in children with SAR to grass pollen and that the combination of Xolair plus SIT-grass is superior to SIT-grass alone. It is concluded, that the combination of Xolair plus SIT demonstrates benefits over and above SIT alone.

Additional assays measuring surrogate markers for activation of mast cells (tryptase) and eosinophils (ECP) provide substantial evidence for suppression of these cells under omalizumab treatment, supporting the clinical results above (see table 6).

TABLE 6
Markers of activation of mast cells (tryptase)
and eosinophils (ECP).

| | | | Birch | Grass | End of |
|---|---|---|---|---|---|
| | | Baseline | season | season | study |
| ECP [%] | omalizumab | 100 | 115 | 128 | 57 |
| | n = 31 | | | | |
| ECP [%] | placebo n = 24 | 100 | 406 | 466 | 207 |
| Tryptase [%] | omalizumab | 100 | 44 | 60 | 53 |
| | n = 31 | | | | |
| Tryptase [%] | placebo n = 24 | 100 | 114 | 115 | 138 |

Safety and Tolerability Results

Treatment was well tolerated compared to SIT alone. In particular no case of anaphylaxis, generalized urticaria or wheezing following injection appeared. Injection site reactions were not different in both groups, SIT alone or SIT plus omalizumab.

Localized urticaria were reported in 2 Instances, both occurring in the omalizumab group. Both were of moderate severity. One was judged to be non-study drug related and resulted in treatment with systemic antihistamine (cetirizine). One case was considered to be study drug related, lasted 24 hr. and ceased without additional treatment.

The frequency of adverse events (AEs, treatment emergent AE, i.e. start of AE at day of or after date of first administration of omalizumab/placebo) was the same in the placebo group (79.63% of patients) and the omalizumab group (79.82% of patients); The most frequently affected body systems (≧5% of patients in either treatment group) are reported in Table 7 and 8 below. The differences in frequency between the two treatments were small, with the exception of nervous system disorders (omalizumab 27.2% vs. placebo 25.0%) and in all cases but one (skin and subcutaneous tissue disorders: omalizumab 13.2% vs. placebo 20.4%) were in favor of omalizumab.

TABLE 7
Study D01. Number (%) of patients with treatment
emergent adverse events (AEs), by body system
(≧5% in either treatment group, safety sample)

| | Omalizumab | Placebo |
|---|---|---|
| | N (%) | N (%) |
| Total number of patients studied | 114 | 108 |
| Total number of patients with an AE | 91 (79.8) | 86 (79.6) |
| Body system affected | | |
| Infections and Infestations | 53 (46.5) | 53 (49.1) |
| Respiratory, thoracic and mediastinal disorders | 38 (33.3) | 46 (42.6) |
| General disorders and administration site | 33 (29.0) | 26 (24.1) |
| conditions | | |
| Nervous system disorders | 31 (27.2) | 27 (25.0) |
| Gastrointestinal disorders | 30 (26.3) | 20 (18.5) |
| Skin & subcutaneous tissue disorders | 15 (13.2) | 22 (20.4) |
| Ear and Labyrinth Disorders | 8 (7.0) | 3 (2.8) |

*Source: Clinical Study report in progress.

TABLE 8
Study D01. Number (%) of patients with treatment emergent adverse events
(AEs), by preferred term (≧5% in either treatment group, safety sample)

| | | Omalizumab | Placebo |
|---|---|---|---|
| | | N (%) | N (%) |
| | Total number of patients | 114 | 108 |
| | studied | | |
| | Total number of patients | 91 (79.8) | 86 (79.6) |
| | with an AE | | |
| Body System | Preferred term AE | | |
| Infections and infestations | Upper respiratory tract | 17 (14.9) | 13 (12.0) |
| | infection | | |
| | Nasopharyngitis | 16 (14.0) | 14 (13.0) |
| | Influenza | 1 (0.9) | 7 (6.5) |
| Respiratory, thoracic and | Asthma | 2 (1.8) | 7 (6.5) |
| | Cough | 30 (26.3) | 25 (23.2) |
| | Dyspnea | 6 (5.3) | 5 (4.6) |
| | Rhinitis | 2 (1.8) | 9 (8.3) |
| General Disorders and administration | Injection site edema | 6 (5.3) | 4 (3.7) |
| site conditions | Injection site pain | 7 (6.1) | 2 (1.9) |
| | Injection site pruritus | 5 (4.4) | 6 (5.6) |
| | Peripheral swelling | 6 (5.3) | 4 (3.7) |
| | Injection site reaction | 7 (6.1) | 1 (0.9) |
| | Pyrexia | 6 (5.3) | 5 (4.6) |
| Nervous system disorders | Headache | 29 (25.4) | 25 (23.2) |
| Gastrointestinal disorders | Sore Throat | 16 (14.0) | 7 (6.5) |
| | Diarrhea | 7 (6.1) | 6 (5.6) |
| Skin and subcutaneous tissue | Eczema | 0 (0) | 7 (6.5) |
| disorders | | | |
| Ear and Labyrinth disorders | Earache | 6 (5.3) | 3 (2.8) |

*A patient with multiple occurrences of one AE under one treatment is counted only once in the AE category for that treatment. A patient with multiple adverse events within a primary system organ class is counted only once in the total row. Source Clinical Study report in progress.

From a safety and tolerability perspective, the incidence of adverse events (AEs) was similar in the Xolair/SIT and in the placebo/SIT groups; injection site reactions (expected in SIT) were more frequent and more pronounced in the placebo/SIT group.

### Example 2

### Combined Effect of Omalizumab and Specific Immunotherapy on In Vitro Leukotriene Release

The population of this analysis is that of the study D01 as described above.

Blood samples taken before and after treatment were used for separation of leukocytes. After pre-stimulation with IL-3 the cells were exposed to grass and birch pollen allergens. In the supernatants SLT (LTC4, LTD4, LTE4) were measured using ELISA (CAST, DPC-Biermann, Germany). Basal SLT release was subtracted from stimulated release beforehand.

Results: Before treatment SLT release to birch and grass pollen exposure did not differ significantly between the four groups. After treatment SLT release to birch pollen was lower in the treated group compared with the control group (Table 9). Similarly SLT release to grass pollen was lower in the treated group compared with the control group.

TABLE 9
IN VITRO LEUKOTRIENE RELEASE

| | | SLT | SLT | |
|---|---|---|---|---|
| Treatment | n | median | (5-95% value) | p-value |
| Omalizumab + SIT-birch | 22 | 101 ng/l | 1-2020 ng/l | 0.0001 |
| Placebo + SIT-birch | 22 | 2905 ng/l | 97-5670 ng/l | |
| Omalizumab + SIT-grass | 23 | 734 ng/l | 1-4673 ng/l | 0.004 |
| Placebo + SIT-grass | 24 | 2835 ng/l | 384-6763 ng/l | |

It can be concluded that, compared to exclusive SIT with pollen allergens, the combination of SIT and omalizumab is associated with a reduced in vitro SLT release after stimulation with allergens. These in vitro results correlate with the clinical results as reported in example 1.

1. A method of treating a subject having an IgE associated disorder comprising administering to the subject an amount of a first composition comprising an immunogenic antigen and administering to the subject an amount of a second composition that inhibits the activity of IgE.

2. The method according to claim 1, wherein the antigen is capable of eliciting or modulating an immune response in a human being.

3. The method according to claim 1, wherein the antigen is an allergen.

4. The method according to claim 3, wherein the allergen is administered in an amount sufficient to induce desensitization to the allergen.

5. The method according to claim 3, wherein the allergen is an aeroallergen.

6. The method according to claim 5, wherein the aeorallergen is an grass pollen.

7. The method according to claim 1, wherein the median symptom load is reduced by at least 10%, by at least 20% or even by at least 40%.

8. The method according to claim 1, wherein the days with intake of any allergy medication are reduced by at least 10%, by at least 20% or by at least 60%.

9. The method according to claim 1, wherein the median use of rescue medication is reduced by at least 10%, by at least 20% or by at least 60%.

10. The method according to claim 1, wherein the IgE associated disorder is an allergy or allergy-related disorder.

11. The method according to claim 10, wherein the IgE associated disorder is SAR.

12. The method according to claim 1, wherein the patient has an age of 6-17 years.

13. The method according to claim 10, wherein the IgE associated disorder is allergic asthma.

14. The method according to claim 1, wherein the composition that inhibits the activity of IgE comprises an anti-IgE antibody.

15. The method according to claim 14, wherein the anti-IgE antibody is a humanized murine antibody.

16. The method according to claim 15, wherein the anti-IgE antibody is Omalizumab.

17. The method according to claim 1, wherein the first composition is administered before the second composition.

18. The method according to claim 1, wherein the first composition is administered with the second composition.

19. The method according to claim 1, wherein in a first treatment period the first composition is titrated up to a maintenance dose, and in a second treatment period the second composition is administered in addition to the maintenance dose of the first composition.

20. The method according to claim 1, wherein the efficacy of treatment is monitored by the measurement of one or more suitable surrogate markers during the treatment period.

21 (canceledl):

22. A pharmaceutical composition comprising an immunogenic antigen and a composition that inhibits the activity of IgE as a combined preparation for simultaneous, separate or sequential use in the therapy of an IgE associated disorder.

23. A pharmaceutical formulation comprising a composition that inhibits the activity of IgE and a composition comprising an immunogenic antigen.

24. A method of treating an allergic response to an antigen or allergy-related disorder during antigen-specific immunotherapy of a subject comprising administering to the subject an amount of a first composition that inhibits the activity of IgE sufficient to decrease the activity of IgE in the subject and administering to the subject a second composition comprising an amount of the antigen sufficient to modulate the immune response to the antigen.

25. The method of claim 24, wherein the composition that inhibits the activity of IgE comprises an anti-IgE antibody.

26. A composition comprising an antigen for use in immunotherapy according to claim 24, wherein the antigen is at a concentration higher than acceptable for use in allergy desensitization therapy.

27. A kit comprising the composition of claim 26 in suitable packaging with instruction for proper use.
